(19) United States
(12) Patent Application Publication
(10) Pub. No.: US 2025/0295428 A1
(43) Pub. Date: Sep. 25, 2025

(54) METHODS AND DEVICES FOR CREATING CENTERED ARTERIOTOMY THROUGH CONDUIT

(71) Applicant: Silk Road Medical, Inc., Sunnyvale, CA (US)

(72) Inventors: Zack Tegels, Sunnyvale, CA (US); Peter Soliman, Sunnyvale, CA (US); Adam Wigginton, Sunnyvale, CA (US); Kristen Lipschultz, Sunnyvale, CA (US); Brad Steele, Sunnyvale, CA (US)

(73) Assignee: Silk Road Medical, Inc., Sunnyvale, CA (US)

(21) Appl. No.: 18/992,667

(22) PCT Filed: Jul. 11, 2023
(86) PCT No.: PCT/US2023/027339

§ 371 Date: Jan. 9, 2025

Related U.S. Application Data
(60) Provisional application No. 63/388,513, filed on Jul. 12, 2022.

(51) Int. Cl. A61B 17/3205; A61B 17/00; A61B 17/32; A61B 17/34

(52) U.S. Cl. CPC A61B 17/32053; A61B 17/00234; A61B 17/3417; A61B 2017/00238; A61B 2017/00353; A61B 2017/00557; A61B 2017/320064

(57) ABSTRACT

Disclosed are various embodiments and related methods of an arteriotomy formation system that can include a center puncturing device and an arteriotomy formation device. The center puncturing device can include a puncturing element that forms a puncture along a wall of a first vessel at a position central to a conduit passageway of a conduit. The arteriotomy formation device can include a tissue fixation element, a tissue capturing element, and a tissue cutting element. The arteriotomy formation device can form an arteriotomy along the first vessel such that the arteriotomy is centered relative to the conduit passageway.

### CROSS-REFERENCE TO RELATED APPLICATION

The current application claims priority under 35 U.S.C. § 119(e) to U.S. Provisional patent application Ser. No. 63/388,513, filed on Jul. 12, 2022, and entitled “METHODS AND DEVICES FOR CREATING CENTERED ARTERIOTOMY THROUGH CONDUIT”, which is incorporated by reference herein in its entirety.

### BACKGROUND

When attaching an anastomosis to a vessel, the vessel can be clamped distal and proximal to an intended access site into the vessel. Such clamping can cause complete cessation of blood flow for the length of time needed (e.g., approximately 10 minutes to 35 minutes) to attach a conduit to the vessel, such as at the intended access site. This can result in potential for vessel damage, vessel dissection, and patient discomfort. In patients with a compromised collateral circulation, complete blood flow cessation may not be tolerated.

Additionally, if an arteriotomy is created prior to attachment of an anastomosis, the arteriotomy may not be centered in the internal diameter of the anastomosis. This may lead to poor tracking of devices due to friction with the arteriotomy, disruption of the anastomosis, and/or enlargement of the arteriotomy outside of the anastomosis suture line. Furthermore, it can be challenging to safely and effectively form an arteriotomy along a vessel via an attached anastomosis. As such, methods and devices to overcome issues related to arteriotomy and anastomosis related procedures are desired.

### SUMMARY

Aspects of the current subject matter described herein relates to embodiments and related methods of an arteriotomy formation system. In one aspect, an arteriotomy formation system is described that includes a center puncturing device. The center puncturing device can include an elongated cylindrical body having an outer wall that slidably engages an inner conduit wall of a conduit mated to a first vessel. The elongated cylindrical body can include a centering passageway extending along a longitudinal axis of the elongated cylindrical body. The center puncturing device can further include a puncturing element that is movable along the centering passageway. The puncturing element can be movable between a retracted configuration and an extended configuration, and a distal end of the puncturing element can be positioned within the centering passageway when in the retracted configuration. The distal end of the puncturing element can extend out of the elongated cylindrical body when in the extended configuration, and the puncturing element can extend along the longitudinal axis of the elongated cylindrical body thereby allowing formation of a puncture along a wall of the first vessel at a position central to a conduit passageway of the conduit.

The arteriotomy formation system can further include an arteriotomy formation device that includes a tissue fixation element including an elongate body having a fixation passageway extending along a length of the tissue fixation element. The arteriotomy formation device can further include a tissue capturing element configured to form a first configuration having a first outer diameter and a second configuration having a second outer diameter that is larger than the first outer diameter. The first configuration can allow the capturing element to pass through the puncture, and the second configuration can allow the tissue capturing element to capture cut out tissue from the wall of the first vessel. The arteriotomy formation device can further include a tissue cutting element that can be moveable relative to the tissue fixation element and the tissue capturing element to form an arteriotomy along the wall of the first vessel, and the tissue cutting element can include an outer cutting wall that slidably engages the inner conduit wall of the conduit and form an arteriotomy along the first vessel such that the arteriotomy is centered relative to the conduit passageway.

In some variations one or more of the following features can optionally be included in any feasible combination. The tissue capturing element can include an inflatable balloon. The inflatable balloon can form a disc shape in the second configuration. The first outer diameter can be smaller than a diameter of the puncture. The tissue cutting element can include a radio-frequency (RF) electrode. The tissue cutting element can include a distal probe configured to perform electrocautery. The tissue cutting element can include at least one expandable blade. The elongated cylindrical body can include a beveled distal end. The conduit can include a sheath or a second vessel. The puncturing element can include a removable guidewire extending therethrough. The capturing element can be configured to slidably travel along the guidewire.

In another interrelated aspect of the current subject matter, a method of an arteriotomy formation system can include forming a puncture along a wall of a first vessel of a patient. The puncture can be formed by a center puncturing device of the arteriotomy formation system at a position that is central to a conduit passageway of a conduit. The center puncturing device can include an elongated cylindrical body including an outer wall that slidably engages an inner conduit wall of the conduit mated to a first vessel, and the elongated cylindrical body can include a centering passageway extending along a longitudinal axis of the elongated cylindrical body. The center puncturing device can include a puncturing element that is movable along the centering passageway, and the puncturing element can be movable between a retracted configuration and an extended configuration. A distal end of the puncturing element can be positioned within the centering passageway when in the retracted configuration, and the distal end of the puncturing element can extend out of the elongated cylindrical body when in the extended configuration. The puncturing element can extend along the longitudinal axis of the elongated cylindrical body thereby allowing formation of the puncture along the wall of the first vessel at a position central to the conduit passageway of the conduit.

The method can further include forming an arteriotomy along the wall of the first vessel. The arteriotomy can be formed by an arteriotomy formation device at a position that is centered relative to the conduit passageway of the conduit. The arteriotomy formation device can include a tissue fixation element including an elongate body having a fixation passageway extending along a length of the tissue fixation element and a tissue capturing element configured to form a first configuration having a first outer diameter and a second configuration having a second outer diameter that is larger than the first outer diameter. The first configuration can allow the capturing element to pass through the puncture and the second configuration can allow the tissue capturing element to capture cut out tissue from the wall of the first vessel. The arteriotomy formation device can include a tissue cutting element that is moveable relative to the tissue fixation element and the tissue capturing element to form an arteriotomy along the wall of the first vessel, and the tissue cutting element can include an outer cutting wall that slidably engages the inner conduit wall of the conduit and forms an arteriotomy along the first vessel such that the arteriotomy is centered relative to the conduit passageway.

In some variations one or more of the following features can optionally be included in any feasible combination. The method can further include capturing, with the tissue capturing element, tissue that has been cut from the wall of the first vessel and removing the captured tissue from the patient. The tissue capturing element can include an inflatable balloon. The inflatable balloon can form a disc shape in the second configuration. The first outer diameter can be smaller than a diameter of the puncture. The tissue cutting element can include a radio-frequency (RF) electrode. The tissue cutting element can include a distal probe configured to perform electrocautery. The tissue cutting element can include at least one expandable blade. The elongated cylindrical body can include a beveled distal end. The conduit can include a sheath or a second vessel. The puncturing element can include a removable guidewire extending therethrough. The capturing element can be configured to slidably travel along the guidewire.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a side view of an embodiment of a center puncturing device of an arteriotomy formation system.

FIG. 2A illustrates a side cross-section view of a part of an example arteriotomy procedure including a conduit attached to a vessel.

FIG. 2B illustrates a side cross-section view of a part of an example arteriotomy procedure including a distal part of the center puncturing device of FIG. 1 extending along the conduit.

FIG. 2C illustrates a side cross-section view of an example part of an arteriotomy procedure including a puncturing element of the center puncturing device of FIG. 1 forming a puncture along the vessel wall.

FIG. 3A illustrates a partial cross-section view of an embodiment of an arteriotomy formation device of the arteriotomy formation system showing a balloon of a tissue capturing element in a deflated configuration.

FIG. 3B illustrates a partial side cross-section view of the arteriotomy formation device of FIG. 3A showing the balloon in an inflated configuration.

FIG. 4A illustrates a side cross-section view of a part of an example arteriotomy procedure showing the distal part of the arteriotomy formation device of FIG. 3A extending along the conduit and the balloon passed through the puncture in a deflated configuration.

FIG. 4B illustrates a side cross-section view of a part of an example arteriotomy procedure showing the distal part of the arteriotomy formation device of FIG. 3A extending along the conduit and the balloon in an inflated configuration.

FIG. 4C illustrates a side cross-section view of a part of an example arteriotomy procedure showing the distal part of the arteriotomy formation device of FIG. 3A extending along the conduit and a tissue cutting element advanced through a vessel wall towards the inflated balloon.

FIG. 4D illustrates a side cross-section view of a part of an example arteriotomy procedure showing an arteriotomy along the vessel wall.

FIG. 5 illustrates a partial side cross-section view of an embodiment of the arteriotomy formation device positioned within a conduit that is not attached to the vessel.

FIG. 6 illustrates another embodiment of the tissue cutting element including a radio-frequency (RF) electrode.

FIG. 7 illustrates another embodiment of the tissue cutting element including a distal probe for performing cauterization.

FIG. 8 illustrates an embodiment of a tissue fixation element including another embodiment of the tissue cutting element that includes an expandable blade element.

Where practical, the same or similar reference numbers denote the same or similar or equivalent structures, features, aspects, or elements, in accordance with one or more embodiments.

### DETAILED DESCRIPTION

The disclosed methods, apparatus, and systems are directed to efficiently and effectively forming an arteriotomy, such as during an anastomosis related procedure. For example, various embodiments of an arteriotomy formation system including a center puncturing device and an arteriotomy formation device are disclosed. The center puncturing device can assist with extending along a conduit and forming a puncture along a vessel wall that is approximately centered relative to a passageway of the conduit. For example, the arteriotomy formation device can utilize the centered puncture formed by the center puncturing device to form a centered arteriotomy (e.g., an enlarged vessel wall opening compared to the puncture) relative to the passageway of the conduit. The arteriotomy formation system can efficiently and consistently form a centered arteriotomy (e.g., relative to an adjacent conduit) that is of a known and desired size. As such, the arteriotomy formation system can minimize bleeding risk and tissue damage, maximize ease of device tracking (e.g., due to reduced friction with tissue surrounding the arteriotomy), and maximize the ability to shunt flow to minimize embolic events. Other benefits associated with the arteriotomy formation system can include the ability to minimize or prevent damage to the formed anastomosis due to the properly sized and centered anastomosis, such as prevent enlargement of the arteriotomy outside of an anastomosis suture line.

Furthermore, the arteriotomy formation system embodiments disclosed herein can form an arteriotomy after attachment of an anastomosis or conduit. This can at least reduce the amount of time required for complete cessation of blood flow in one or more parts of the body and thus reduce potential vessel damage and/or allow patients with compromised circulations to undergo anastomosis procedures.

In some embodiments, the arteriotomy formation system, including the center puncturing device and the arteriotomy formation device, can be sized to assist with performing an arteriotomy associated with a variety of procedures, including anastomosis procedures associated with various parts of the body. As such, the arteriotomy formation system can be used with a variety of vasculature, including the common femoral, subclavian, carotid, jugular, inferior vena cava, aorta, apex of left or right ventricle, etc., without departing from the scope of this disclosure. Furthermore, the arteriotomy formation system can be used with one or more of a variety of access systems and sheaths.

Various embodiments of an arteriotomy formation system that can include a center puncturing device and an arteriotomy forming device are described below along with related methods of use in example arteriotomy and anastomosis procedures.

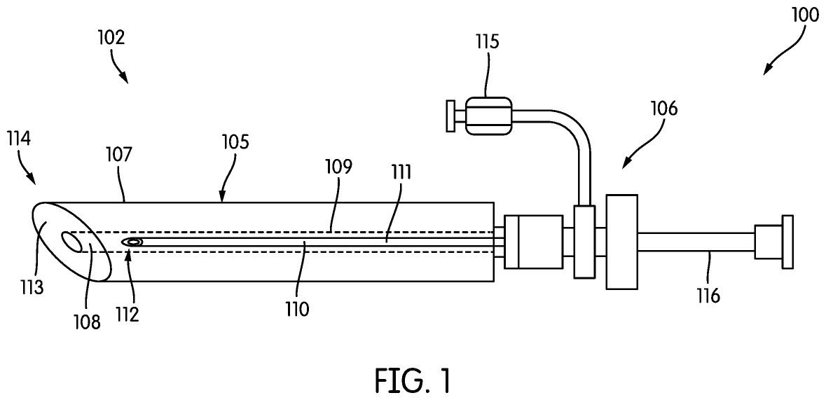

FIG. 1 illustrates an embodiment of a center puncturing device 102 of an arteriotomy formation system 100. The center puncturing device 102 can be configured to be inserted into a proximal end of a conduit, such as a conduit connected to a vessel wall. For example, the center puncturing device 102 can extend along a length of a conduit passageway of the conduit and form a puncture through the vessel wall. The formed puncture can extend approximately along a longitudinal axis of the center puncturing device and the conduit. As will be disclosed in greater detail below, the centered puncture can provide a guide for efficiently and effectively forming an arteriotomy of a desired shape and size along the vessel wall that aligns centrally with the conduit passageway.

As shown in FIG. 1, the center puncturing device 102 can include an elongated cylindrical body 105 that extends from a coupling end 106 of the center puncturing device 102. For example, the elongated cylindrical body 105 can include an outer wall 107 having a diameter that is the same as or slightly smaller (e.g., approximately 1 millimeter smaller diameter) than an inner wall diameter of a conduit along which the elongated cylindrical body 105 can extend along for forming a puncture. For example, the outer wall 107 of the elongated cylindrical body 105 can have a diameter that allows the outer wall 107 to have approximately a sliding fit or sliding engagement with the inner wall of the conduit. The sliding fit between the outer wall 107 of the elongated cylindrical body 105 and the inner wall of the conduit can allow the elongated cylindrical body 105 to travel along the conduit, such as to position a distal end of the elongated cylindrical body 105 at or adjacent a distal end of the conduit while also centering the elongated cylindrical body 105 relative to the conduit. By centering the elongated body 105 relative to the conduit, the center puncturing device 102 can efficiently and effectively form a puncture through a vessel wall at a location that is approximately at a center point of a distal opening of the conduit.

As shown in FIG. 1, the elongated cylindrical body 105 can include a centering passageway 108 that includes a passageway longitudinal axis that is collinear with a longitudinal axis of the elongated cylindrical body 105. As such, the centering passageway 108 extends centrally along the elongated cylindrical body 105, as shown in FIG. 1. The center puncturing device 102 can also include a puncturing element 110 that is moveable along the centering passageway 108. The puncturing element 110 can have an elongated puncturing body 111 with a distal end 112 that is sharp and/or beveled for piercing through tissue, such as through a vessel wall. The elongated puncturing body 111 can have an outer diameter that is smaller than an inner wall 109 of the centering passageway 108. In some embodiments, the puncturing element 110 can form a sliding fit (e.g., sliding engagement) with the inner wall 109 of the centering passageway 108 thereby centering the puncturing element 110 relative to the elongated cylindrical body 105.

The puncturing element 110 can be movable between a retracted configuration and an extended configuration. In the retracted configuration, a distal end 112 of the puncturing element 110 can be positioned within the centering passageway 108. In the extended configuration, the distal end 112 of the puncturing element 110 can extend out of the elongated cylindrical body 105, such as for forming the centered puncture relative to the centering passageway.

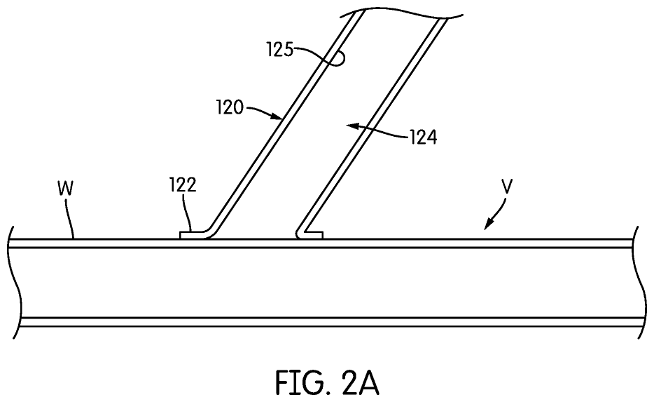

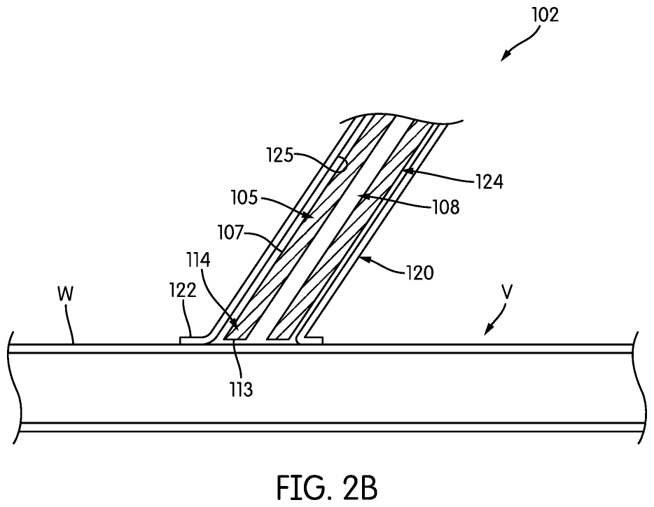

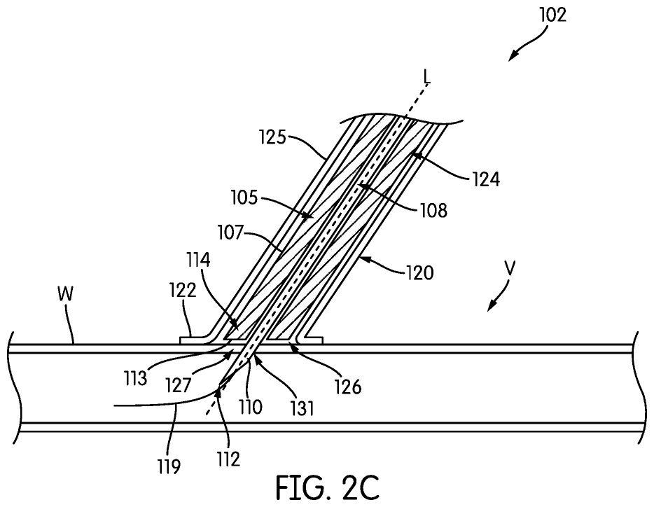

In some embodiments, a distal end 114 of the elongated cylindrical body 105 includes a distal surface 113 that is positioned at an angle relative to the longitudinal axis of the elongated cylindrical body 105, thereby forming an angled or beveled shape at the distal end 114 of the elongated cylindrical body 105. The angled or beveled shape can allow the distal surface 113 of the puncturing element 110 to substantially contact a vessel wall and align the elongated cylindrical body 105 at an angle relative to the vessel wall, such as at a same or similar angle the conduit extends (relative to the mated vessel). Other shapes and features can be included at the distal end 114 of the elongated cylindrical body 105, such as the distal surface 113 being perpendicular to the longitudinal axis of the elongated cylindrical body 105, without departing from the scope of this disclosure. For example, the elongated cylindrical body 105 can have an outer wall diameter that is approximately the same as or approximately 1 millimeter smaller than a conduit (e.g., conduit 120 in FIG. 2B) along which the elongated cylindrical body 105 extends along. In some embodiments, the elongated cylindrical body 105 can have a length of approximately 5 centimeters to approximately 35 centimeters, which can depend on a length of the conduit.

In some embodiments, the coupling end 106 can include at least one valve 115 for controlling a fluid line that can be in communication with the center puncturing device. For example, the valve 115 can include one or more of a hemostasis valve, rotating hemostasis valve (RHV), Tuohy Borst valve, luer lock, and/or other similar valves. As shown in FIG. 1, the coupling end 106 can include an extension 116 that can provide a working passageway and/or control movement of the puncture element 110 along the centering passageway 108. For example, the extension 116 can be advanced distally to move the puncturing element 110 into the extended configuration and retracted proximally to move the puncturing element 110 into the retracted configuration. Other features and couplings can be included in the coupling end 106 of the center puncturing device 102 without departing from the scope of this disclosure.

FIGS. 2A-2D illustrate a part of an example arteriotomy procedure using the center puncturing device 102. As shown in FIG. 2A, a conduit 120 can be attached to a wall W of a vessel V, such as a distal conduit end 122 of the conduit 120 can be sutured to the wall W of the vessel V. For example, the conduit 120 can be another vessel (e.g., formed of tissue). In some embodiments, the conduit 120 can be a sheath and/or formed out of one or more of a variety of materials, including biocompatible materials. As shown in FIG. 2A, the distal conduit end 122 of the conduit 120 can be securely attached to the vessel V, such as using suture. In some embodiments, the conduit 120 can be unattached to the vessel V and, instead, the distal conduit end 122 can be positioned against or adjacent the vessel V. As shown in FIG. 2A, the conduit 120 can include a conduit passageway 124 and an inner conduit wall 125 defining the conduit passageway 124.

After the distal conduit end 122 is positioned at and/or secured to a desired location along the wall W of the vessel V, the elongated cylindrical body 105 of the center puncturing device 102 can be inserted and advanced along the conduit passageway 124. As shown in FIG. 2B, the distal end 114 of the elongated cylindrical body 105 can be advanced along the conduit 120 until the distal surface 113 contacts and/or is adjacent to the wall W of the vessel V. The distal surface 113 can be angled relative to the longitudinal axis of the elongated cylindrical body 105 such that the elongated cylindrical body 105 can extend along a length of the conduit 120 that is coupled to the vessel V at an angle while allowing the distal surface 113 to align approximately parallel to the vessel wall W. This can allow the distal end 112 of the puncturing element 110 to efficiently and effectively align with a longitudinal axis of the conduit 120 and form a puncture 131 through the vessel wall along the longitudinal axis of the conduit 120, as shown in FIG. 2C.

In some embodiments, the distal opening 126 of the conduit 120 can be defined by the inner conduit wall 122 at or along a distal conduit end 122. As shown in FIG. 2C, the distal opening can define a vessel wall area 127 along the wall W of the vessel V that is in communication with the conduit passageway 124. As such, in some embodiments the vessel wall area 127 can be defined by the distal conduit end 122. In order to reduce friction and damage to the wall W of the vessel V, a puncture and/or arteriotomy can be formed at a location that is center to the distal opening 126 of the conduit 120.

As shown in FIG. 2C, a guidewire 119 can be extended out from the distal end 112 of the puncturing element 110 after the distal end 112 of the puncturing element 110 is passed through the wall W of the vessel V. After the puncture 131 is formed through the wall W of the vessel V, the center puncturing device 102 can be removed from the conduit 120. In some embodiments, the guidewire 119 is left to extend through the puncture 131 to assist with guiding one or more features of an embodiment of the arteriotomy formation device to the location of the puncture 131, such as for forming the arteriotomy.

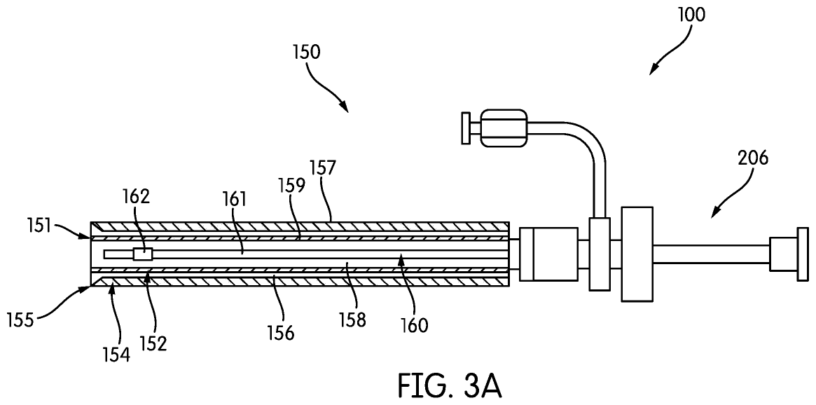

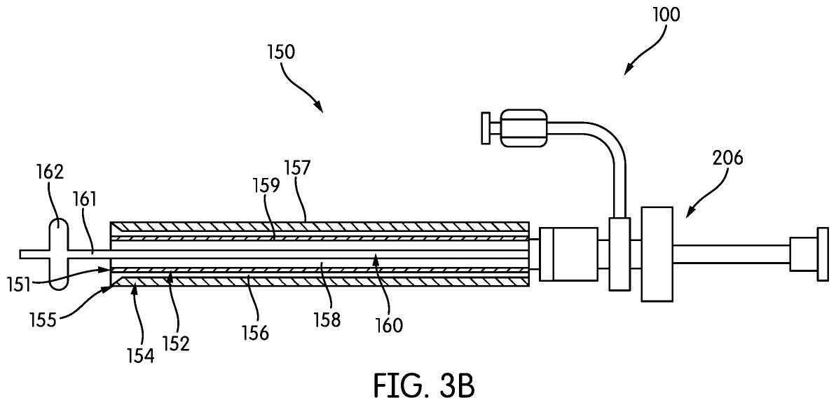

FIGS. 3A and 3B illustrate an embodiment of the arteriotomy formation device 150 of the arteriotomy formation system 100 for forming an arteriotomy that is centered relative to a conduit (e.g., anastomosis), which can at least ensure a reduction in device tracking, tissue damage, and damage caused by emboli.

As shown in FIGS. 3A and 3B, the arteriotomy formation device 150 can include a tissue fixation element 152 that is configured to contact a part of the vessel wall (e.g., vessel wall area 127). In some embodiments, the tissue fixation element 152 can have an elongated tubular shape and can be formed out of a material that is sufficiently rigid to allow the tissue fixation element 152 to apply a force against the vessel wall area 127. As shown in FIG. 3A, the tissue fixation element 152 can extend from an embodiment of the coupling end 206 of the arteriotomy formation device 150. The coupling end 206 of the arteriotomy formation device 150 can include at least some of the features described above with respect to the coupling end 106 of FIG. 1. In some embodiments, the tissue fixation element 152 can be movable along a longitudinal axis of the arteriotomy formation device 150, such as relative to a part of the coupling end 106. For example, the tissue fixation element 152 can be made out of a metal or plastic material, such as one or more of a stainless steel and a biocompatible material.

As shown in FIGS. 3A and 3B, the arteriotomy formation device 150 can also include a tissue cutting element 154 that includes a distal cutting end 155 configured to cut through a wall of a vessel to form an arteriotomy. For example, the tissue cutting element 154 can have an elongated tubular body with a sharp and/or beveled distal cutting end 155. In some embodiments, the tissue cutting element 154 can be movable along a longitudinal axis of the arteriotomy formation device 150, such as relative to a part of the coupling end 206 and/or the tissue fixation element 152. As shown in FIG. 3A, the tissue cutting element 154 can have an inner pathway 156 having a diameter that is larger than the tissue fixation element 152 such that the tissue fixation element 152 is positioned along the inner pathway 156.

In some embodiments, the distal cutting end 155 of the tissue cutting element 154 can have a circular shape, thereby forming a circular shaped arteriotomy along the vessel wall. Other shaped distal cutting ends 155 are within the scope of this disclosure. In some embodiments, the tissue cutting element 154 can include an outer cutting wall 157 having a diameter that is smaller than a diameter of a conduit along which the tissue cutting element 154 is to be extended along. For example, the outer cutting wall 157 can have a sliding fit or sliding engagement with an inner conduit wall (e.g., the inner conduit wall 125 shown, for example, in FIG. 2A) to thereby allow at least the tissue cutting element 154 to be positioned central to the surrounding conduit. This can allow elements positioned within the tissue cutting element 154, such as the tissue fixation element 152, to also be positioned central to the surrounding conduit.

For example, the tissue cutting element 154 can be made out of a metal or plastic material, such as one or more of a stainless steel and a biocompatible material. The size of the tissue cutting element 154 can be dependent upon a conduit size used in a procedure. For example, in some embodiments the tissue cutting element 154 can have an outer diameter that is approximately 3 millimeters to approximately 10 millimeters. In some embodiments, the tissue cutting element 154 can have an inner diameter that is approximately 2.5 millimeters to approximately 9.5 millimeters.

As shown in FIG. 3A, the tissue fixation element 152 can include a fixation pathway 158 that allows a capturing element 160 to extend therealong. In some embodiments, the capturing element 160 can include a balloon catheter 161 with an inflatable balloon 162 positioned adjacent a distal end of the balloon catheter 161. For example, the balloon catheter 161 can include a fluid line that allows a fluid source in communication with the coupling end 206 to provide a fluid (e.g., air) to inflate the balloon 162 to form an inflated configuration, as shown in FIG. 3B. For example, the inflated balloon 162 can have a disc shape, as shown in FIG. 3B, however, other shapes are within the scope of this disclosure. Additionally, the fluid line along the balloon catheter 161 can allow fluid to be removed from the balloon 162 to deflate the balloon 162 to form the deflated configuration, as shown in FIG. 3A.

In some embodiments, the capturing element 160 can be movable along a longitudinal axis of the arteriotomy formation device 150, such as relative to a part of the coupling end 206 and/or the tissue fixation element 152. The capturing element 160 can include a variety of shapes and sizes. For example, the balloon 162 can be sized based upon the size of tissue cutting element 154. In some embodiments, the balloon 162 can have a diameter (when inflated) of approximately 3 millimeters to approximately 10 millimeters. In some embodiments, the balloon catheter 161 can have an outer diameter that is approximately 0.035 inch to approximately 0.040 inch. For example, the capturing element 160 can be made out of one or more of an elastic material, a plastic material, polyurethane, silicone, and a biocompatible material. Although the capturing element 160 is described as having an inflatable balloon 162, the capturing element 160 can include or more of a variety of features for assisting with capturing tissue.

In some embodiments, the balloon catheter 161 of the capturing element 160 can have an outer diameter that is smaller than a diameter of an inner fixation wall 159 defining the fixation pathway 158 of the tissue fixation element 152. For example, the outer diameter of the balloon catheter 161 can be sized such that the balloon catheter 161 has approximately a sliding fit with the inner fixation wall 159 of the tissue fixation element 152. As such, the tissue fixation element 152 can assist with guiding the balloon 162 and distal end of the balloon catheter along the longitudinal axis of the tissue cutting element 154, as well as the surrounding conduit), thereby allowing the distal end of the balloon catheter 161 and the balloon 162 to pass through the puncture 131 made along the vessel wall for forming an arteriotomy that is central to the distal opening 126 of the conduit 120. In some embodiments, the balloon catheter 161 can include a guidewire pathway that allows the balloon catheter 161 to slidably travel along a guidewire, such as the guidewire 119 extending through the puncture 131.

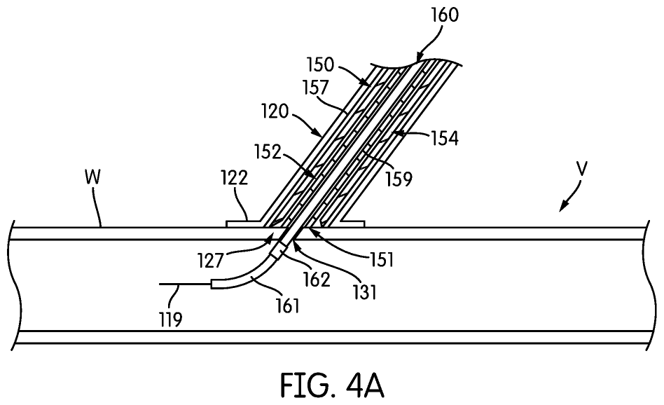

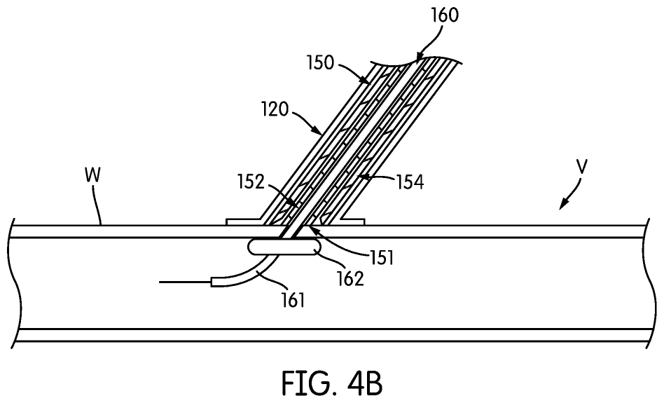

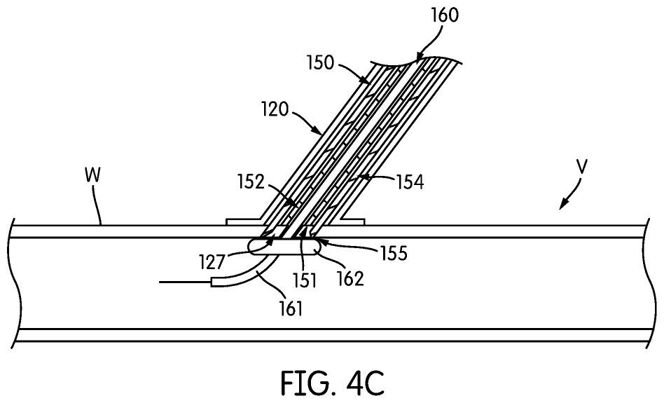

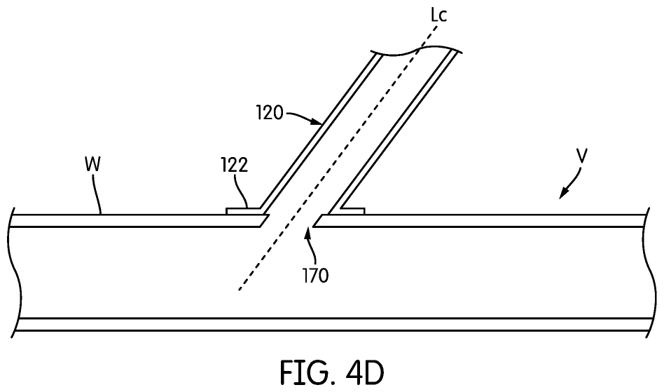

FIGS. 4A-4D illustrate a part of an example arteriotomy procedure using the arteriotomy formation device 150, such as for forming an arteriotomy that is central to the distal opening 126 of the conduit 120. As shown in FIG. 4A, the distal part of the arteriotomy formation device 150 can be inserted into and extended along a length of the conduit 120. For example, the arteriotomy formation device 150 can be advanced along the conduit passageway 124 (e.g., along the guidewire 119) until a distal end of the arteriotomy formation device 150 is positioned at or adjacent the wall W of the vessel V. For example, a distal fixation end 151 of the tissue fixation element 152 and/or the distal cutting end 155 of the tissue cutting element 154 can be placed in contact and/or adjacent the wall W of the vessel V, such as along the vessel wall area 127. After the distal end of the arteriotomy formation device 150 is positioned at or adjacent the wall of the vessel, the capturing element 160 can be advanced distally such that the balloon 162 (in a deflated configuration) can advance through the puncture 131 (e.g., via along the guidewire 119), as shown in FIG. 4A. As disclosed above, the puncture 131 through the wall W of the vessel V can be formed by the center puncturing device 102 and located at approximately a center point of the vessel wall area 127 and/or distal opening 126 of the conduit 120.

As shown in FIG. 4B, the balloon 162 can be filled with a fluid (e.g., via a fluid pathway along the balloon catheter 161) and transition into an inflated configuration. After the balloon 162 is in the inflated configuration, the capturing element 160 can be advanced proximally until the balloon 162 contacts the vessel wall W, as shown in FIG. 4B. In this configuration, at least a part of the vessel wall area 127 is positioned between the balloon 162 and both the tissue cutting element 154 and the tissue fixation element 152. This can allow the balloon 162 and tissue fixation element 152 to retain cut out vessel wall tissue formed during formation of the arteriotomy by the tissue cutting element 154, as will be further described below.

As shown in FIG. 4C, the distal cutting end 155 of the tissue cutting element 154 can be advanced through the vessel wall W to cut out at least a part of the vessel wall area 127. The tissue cutting element 154 can be advanced towards the balloon 162 until the distal cutting end 155 is at or adjacent the balloon 162. The cut out portion of the vessel wall area 127 can be captured between the balloon 162 and the tissue fixation element 152, as well as surrounded by the tissue cutting element 154. As such, the arteriotomy formation device 150 can be retracted and removed from the conduit 120, including the cut out portion of the vessel wall area 127, thereby preventing unwanted embolic events. As shown in FIG. 4D, an arteriotomy 170 along the vessel wall W can be formed and the cut out portion of the vessel wall area 127 can be removed by the arteriotomy formation device 150. Furthermore, as shown in FIG. 4D, the formed arteriotomy 170 can be centered relative to the conduit 120 (e.g., a center of the arteriotomy can be positioned approximately along a longitudinal conduit axis Lc of the conduit).

Other embodiments and related methods of the arteriotomy formation system 100, including the center puncturing device 102 and the arteriotomy formation device 150, are within the scope of this disclosure and some are described in further detail below.

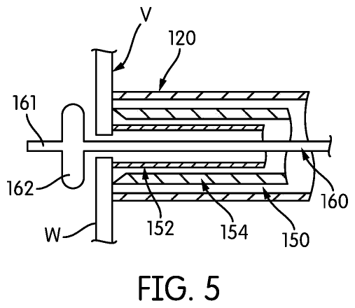

FIG. 5 illustrates an embodiment of the arteriotomy formation device 150, such as the arteriotomy formation device 150 described above with respect to FIGS. 3A and 3B, positioned within a conduit 120 that is not attached to the vessel V. For example, the conduit 120 (e.g., sheath, trocar) can be made out of a metal and/or synthetic material, such as a plastic and/or biocompatible material.

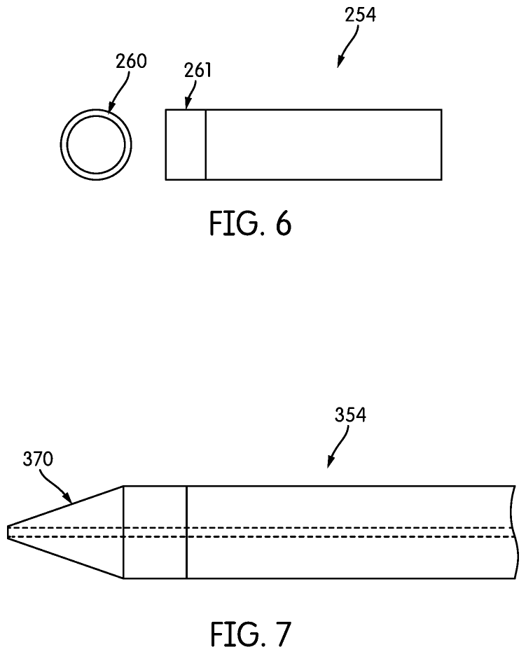

FIG. 6 illustrates another embodiment of the tissue cutting element 254 of the arteriotomy formation device 150. The tissue cutting element 254 illustrated in FIG. 6 can cut tissue using radio-frequency (RF). For example, the tissue cutting element 254 can include an RF electrode 261 at or adjacent an embodiment of the distal cutting end 260 of the tissue cutting element 254. As shown in FIG. 6, the RF electrode can have a circular shape that can form a circular shaped arteriotomy along a vessel wall. Other shaped RF electrodes such as oval or elliptical shaped RF electrodes are also within the scope of this disclosure. During use, the RF electrode 261 of the tissue cutting element 254 can be advanced distally towards and/or through a vessel wall while delivering RF energy to the vessel wall in order to form an arteriotomy along the vessel wall.

FIG. 7 illustrates another embodiment of the tissue cutting element 354 of the arteriotomy formation device 150. The tissue cutting element 354 illustrated in FIG. 7 can cut tissue using electrocautery. For example, the tissue cutting element 254 can include a distal probe 370 that can provide an electric current to burn or destroy tissue, such as vessel wall tissue to form an arteriotomy. As shown in FIG. 7, the distal probe 370 can have a cone or tapered shape that can form a circular shaped arteriotomy along a vessel wall. Other shaped distal probes 370 such as oval or elliptical shaped distal probes 370 are also within the scope of this disclosure. During use, the distal probe 370 of the tissue cutting element 254 can include an electric current and be advanced distally towards and/or through a vessel wall. In some embodiments, the distal probe 370 can burn and destroy vessel wall tissue to form the arteriotomy without forming a cut out piece of tissue for removal. As such, some embodiments of the arteriotomy formation device 150 can be void of the capturing element 160, such as when the tissue cutting element 354 uses electrocautery to form the arteriotomy.

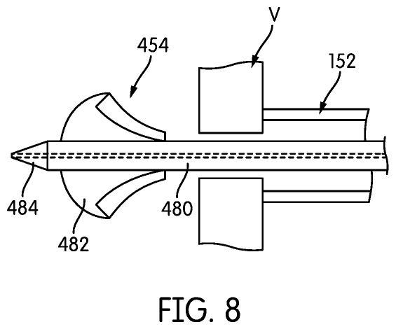

FIG. 8 illustrates another embodiment of the tissue cutting element 454 of the arteriotomy formation device 150. The tissue cutting element 454 illustrated in FIG. 8 can include one or more expandable blades 482 that can form an arteriotomy in a vessel wall. The expandable blades can be positioned along a trocar 484 and form a collapsed configuration when being passed through the vessel wall (e.g., via the puncture 131) and form an expanded configuration when forming the arteriotomy. The expandable blades 482 can extend and/or taper radially away from the trocar 484 such that arteriotomy formation does not include removal of vessel wall tissue. As such, some embodiments of the arteriotomy formation device 150 can be void of the capturing element 160, such as when the tissue cutting element 454 having expandable blades 482 is used to form the arteriotomy. In some embodiments, the one or more expandable blades 482 are spring loaded and retract as the tissue cutting element 454 is pushed through a vessel wall. In some embodiments, the one or more expandable blades 482 are actively controlled, such as by a user, to selectively form the collapsed configuration and the expanded configuration.

Various dimensions of the arteriotomy formation system 100, such as the center puncturing device 102 and/or the arteriotomy formation device 150, can be modified and customized in order to size any part of the arteriotomy formation system 100 for one or more procedures, such as anastomosis and/or arteriotomy procedures associated with one or more of a variety of vasculature and/or conduits. As such, the arteriotomy formation system 100, including the center puncturing device 102 and/or the arteriotomy formation device 150, can be modified and customized to have outer dimensions and inner dimensions that are safe and effective for performing procedures in one or more of a variety of vasculature and conduits.

During any part of a procedure that involves the use of the arteriotomy formation system 100, such as the center puncturing device 102 and/or the arteriotomy formation device 150, the vessel V can be occluded in one or more locations, such as adjacent to the location where the arteriotomy 170 is formed. For example, manual occlusion of the vessel V by a clinician at an occlusion location may be provided from the outside of the vessel V using a vascular clamp, such as a Rummel tourniquet or vessel loop positioned proximal to the sheath insertion site. Occlusion of the vessel V can form a closed section of the vessel along which blood is prevented from flowing. Once the vessel V has been treated by the operator, the vascular clamp can be released permitting resumption of blood flow. One or more of a variety of ways can be used to occlude the vessel V during a procedure without departing from the scope of this disclosure.

While these descriptions contain many specifics, these should not be construed as limitations on the scope of an invention that is claimed or of what may be claimed, but rather as depictions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore, the spirit and scope of the appended claims should not be limited to the description of the embodiments contained herein.

1-23. (canceled)

24. An arteriotomy formation system, comprising:
a center puncturing device, comprising:
an elongated cylindrical body including an outer wall that slidably engages an inner conduit wall of a conduit mated to a first vessel, the elongated cylindrical body including a centering passageway extending along a longitudinal axis of the elongated cylindrical body; and
a puncturing element that is movable along the centering passageway, the puncturing element being movable between a retracted configuration and an extended configuration, a distal end of the puncturing element being positioned within the centering passageway when in the retracted configuration, the distal end of the puncturing element extending out of the elongated cylindrical body when in the extended configuration, the puncturing element extending along the longitudinal axis of the elongated cylindrical body thereby allowing formation of a puncture along a wall of the first vessel at a position central to a conduit passageway of the conduit; and
an arteriotomy formation device, comprising:
a tissue fixation element including an elongate body having a fixation passageway extending along a length of the tissue fixation element;
a tissue capturing element configured to form a first configuration having a first outer diameter and a second configuration having a second outer diameter that is larger than the first outer diameter, the first configuration allowing the capturing element to pass through the puncture and the second configuration allowing the tissue capturing element to capture cut out tissue from the wall of the first vessel; and
a tissue cutting element being moveable relative to the tissue fixation element and the tissue capturing element to form an arteriotomy along the wall of the first vessel, the tissue cutting element including an outer cutting wall that slidably engages the inner conduit wall of the conduit and forms an arteriotomy along the first vessel such that the arteriotomy is centered relative to the conduit passageway.

25. The arteriotomy formation system of claim 24, wherein the tissue capturing element includes an inflatable balloon.

26. The arteriotomy formation system of claim 25, wherein the inflatable balloon forms a disc shape in the second configuration.

27. The arteriotomy formation system of claim 25, wherein the first outer diameter is smaller than a diameter of the puncture.

28. The arteriotomy formation system of claim 24, wherein the tissue cutting element comprises a radio-frequency (RF) electrode.

29. The arteriotomy formation system of claim 24, wherein the tissue cutting element comprises a distal probe configured to perform electrocautery.

30. The arteriotomy formation system of claim 24, wherein the tissue cutting element comprises at least one expandable blade.

31. The arteriotomy formation system of claim 24, wherein the elongated cylindrical body includes a beveled distal end.

32. The arteriotomy formation system of claim 24, wherein the conduit includes a sheath or a second vessel.

33. The arteriotomy formation system of claim 24, wherein the puncturing element includes a removable guidewire extending therethrough.

34. The arteriotomy formation system of claim 33, wherein the capturing element is configured to slidably travel along the guidewire.

35. A system for forming an arteriotomy, the system comprising:
a puncturing device comprising:
a conduit configured to be coupled to a wall of a vessel,
a cylindrical body configured to be advanced within the conduit, the cylindrical body having a centering passageway formed therein, and
a puncturing element configured to be advanced through the centering passageway and into the vessel; and
an arteriotomy formation device, comprising:
a tissue fixation element comprising an elongate body having a fixation passageway formed therein,
a tissue capturing element configured to shift between a first configuration having a first outer diameter and a second configuration having a second outer diameter that is larger than the first outer diameter, and
a tissue cutting element being moveable relative to the tissue fixation element and the tissue capturing element to form an arteriotomy along the wall of the first vessel.

36. The system of claim 35, wherein the tissue capturing element includes an inflatable balloon.

37. The system of claim 35, wherein the tissue cutting element comprises a radio-frequency (RF) electrode.

38. The system of claim 35, wherein the tissue cutting element comprises at least one expandable blade.

39. A method of an arteriotomy formation system, comprising:
forming a puncture along a wall of a first vessel of a patient, the puncture being formed by a center puncturing device of the arteriotomy formation system at a position that is central to a conduit passageway of a conduit, the center puncturing device, comprising:
an elongated cylindrical body including an outer wall that slidably engages an inner conduit wall of the conduit mated to a first vessel, the elongated cylindrical body including a centering passageway extending along a longitudinal axis of the elongated cylindrical body; and
a puncturing element that is movable along the centering passageway, the puncturing element being movable between a retracted configuration and an extended configuration, a distal end of the puncturing element being positioned within the centering passageway when in the retracted configuration, the distal end of the puncturing element extending out of the elongated cylindrical body when in the extended configuration, the puncturing element extending along the longitudinal axis of the elongated cylindrical body thereby allowing formation of the puncture along the wall of the first vessel at a position central to the conduit passageway of the conduit; and
forming an arteriotomy along the wall of the first vessel, the arteriotomy being formed by an arteriotomy formation device at a position that is centered relative to the conduit passageway of the conduit, the arteriotomy formation device comprising:
a tissue fixation element including an elongate body having a fixation passageway extending along a length of the tissue fixation element;
a tissue capturing element configured to form a first configuration having a first outer diameter and a second configuration having a second outer diameter that is larger than the first outer diameter, the first configuration allowing the capturing element to pass through the puncture and the second configuration allowing the tissue capturing element to capture cut out tissue from the wall of the first vessel; and
a tissue cutting element being moveable relative to the tissue fixation element and the tissue capturing element to form an arteriotomy along the wall of the first vessel, the tissue cutting element including an outer cutting wall that slidably engages the inner conduit wall of the conduit and forms an arteriotomy along the first vessel such that the arteriotomy is centered relative to the conduit passageway.

40. The method of claim 39, further comprising:
capturing, with the tissue capturing element, tissue that has been cut from the wall of the first vessel; and
removing the captured tissue from the patient.

41. The method of claim 39, wherein the tissue capturing element includes an inflatable balloon.

42. The method of claim 39, wherein the tissue cutting element comprises a radio-frequency (RF) electrode.

43. The method of claim 39, wherein the tissue cutting element comprises at least one expandable blade.
